(19) United States
(12) Patent Application Publication
(10) Pub. No.: US 2026/0083338 A1
(43) Pub. Date: Mar. 26, 2026

(54) APPARATUS AND METHOD FOR INFERRING STROKE VOLUME VARIATION BASED ON CENTRAL VENOUS PRESSURE WAVEFORM

(71) Applicant: Seoul National University Hospital, Seoul (KR)

(72) Inventors: Insun PARK, Seoul (KR); Jae Hyon PARK, Seoul (KR)

(21) Appl. No.: 19/335,467

(22) Filed: Sep. 22, 2025

(30) Foreign Application Priority Data
Sep. 23, 2024 (KR) 10-2024-0128586
Sep. 16, 2025 (KR) 10-2025-0133055

(51) Int. Cl. A61B 5/021; A61B 5/00; A61B 5/0215

(52) U.S. Cl. CPC A61B 5/02125; A61B 5/02152; A61B 5/7267

(57) ABSTRACT

A stroke volume variation inference apparatus according to an embodiment comprises a memory for storing at least one instruction; and a processor, wherein as the at least one instruction is executed by the processor, central venous pressure (CVP) waveform information is obtained from a catheter inserted into a patient's body, and the central venous pressure waveform information is provided to a pre-trained inference model configured to infer stroke volume variation (SVV) in the heart, such that information about the stroke volume variation for the patient is obtained.

This application claims priority to Korean Patent Application No. 10-2024-0128586, filed on Sep. 23, 2024, and to Korean Patent Application No. 10-2025-0133055, filed on Sep. 16, 2025 the entirety of which is incorporated herein by reference for all purposes.

### TECHNICAL FIELD

An embodiment relates to an apparatus and method for inferring a patient's Stroke Volume Variation (SVV) from a Central Venous Pressure (CVP) waveform.

### BACKGROUND OF THE INVENTION

Central Venous Pressure (CVP) is a representative indicator of cardiac preload. CVP has long been used to evaluate cardiovascular function and circulatory status. CVP is widely used in clinical practice because it can be easily measured through a central venous line. In particular, CVP has been established as a key indicator for determining fluid responsiveness in intensive care units and operating rooms. In fact, there are reports that approximately 73% of anesthesiologists in the United States and approximately 84% of anesthesiologists in Europe still utilize CVP for fluid management.

However, according to previous studies, a limitation has been pointed out that it is difficult to accurately infer fluid responsiveness solely from static CVP values. This is because the CVP value varies between patients and even within the same patient over time, and the same CVP value may exhibit different responsiveness depending on the shape of the Frank-Starling curve. Accordingly, technologies have recently emerged that infer fluid responsiveness with higher accuracy than CVP by using dynamic indicators such as Stroke Volume Variation (SVV), Pulse Pressure Variation (PPV), and Systolic Pressure Variation (SPV). Nevertheless, CVP is still widely used for fluid management worldwide, and its utility is particularly high in high-risk surgery or critical care environments.

### SUMMARY OF THE INVENTION

### Problem to be Solved

An object to be solved according to an embodiment includes inferring an SVV by analyzing CVP waveform data.

Furthermore, achieving an accuracy level similar to that of existing arterial pressure-based equipment by applying a deep learning algorithm (e.g., Long Short-Term Memory (LSTM)) suitable for time-series analysis to the aforementioned analysis may be included in the object of an embodiment.

However, the objects to be solved according to an embodiment are not limited to the technical problems described above, and other technical problems not mentioned will be clearly understood by a person skilled in the art from the following description.

### Solution to the Problem

A deep learning-based central venous pressure waveform stroke volume variation inference apparatus according to a first embodiment comprises a memory for storing at least one instruction; and a processor, wherein as the at least one instruction is executed by the processor, central venous pressure (CVP) waveform information is obtained from a catheter inserted into a patient's body, and the central venous pressure waveform information is provided to a pre-trained inference model configured to infer stroke volume variation (SVV) in the heart, such that information about the stroke volume variation for the patient may be obtained.

Furthermore, the central venous pressure waveform information may be a central venous pressure measured over a predetermined time, represented in a numerical type or a graphical format.

Furthermore, as the at least one instruction is executed by the processor, information about the central venous pressure waveform may be provided to a pre-trained waveform decomposition model, a cardiac component waveform and a respiratory component waveform, which are results of decomposing the central venous pressure waveform information, may be obtained from the waveform decomposition model, and the cardiac component waveform and the respiratory component waveform may be provided to the inference model as the central venous pressure waveform information.

Furthermore, the patient may be a patient breathing using a predetermined ventilator under positive pressure ventilation, and the inference model may infer the information about the stroke volume variation for the patient by further considering information about the type of ventilator the patient is using for respiration.

Furthermore, as the at least one instruction is executed by the processor, information about an insertion site of the catheter in the patient may be additionally obtained, the central venous pressure waveform information is corrected according to the catheter insertion site, and information about the corrected central venous pressure waveform may be provided to the inference model.

Furthermore, as the at least one instruction is executed by the processor, at least one of information on the patient's heart rate or respiratory rate, information on a pressure state of the lungs or thoracic cavity, information on blood components, and information on a position of the patient on a Frank-Starling curve may be additionally obtained, and the inference model may perform inference by considering the additionally obtained information together with the central venous pressure waveform information.

A stroke volume variation inference method according to a second embodiment to be performed by a stroke volume variation inference apparatus and the method comprising: obtaining central venous pressure (CVP) waveform information from a catheter inserted into a patient's body; and obtaining information about stroke volume variation for the patient by providing the central venous pressure waveform information to a pre-trained inference model configured to infer stroke volume variation (SVV) in the heart.

Furthermore, the central venous pressure waveform information may be represented as a central venous pressure measured over a predetermined time, in a numerical type or a graphical format.

Furthermore, the method may further comprise: providing the central venous pressure waveform information to a pre-trained waveform decomposition model; and obtaining a cardiac component waveform and a respiratory component waveform, which are results of decomposing the central venous pressure waveform information, from the waveform decomposition model, wherein the cardiac component waveform and the respiratory component waveform may be provided to the inference model as the central venous pressure waveform information.

Furthermore, the patient may be a patient breathing using a predetermined ventilator under positive pressure ventilation, and the inference model may infer the information about the stroke volume variation for the patient by further considering information about the type of ventilator the patient is using for respiration.

Furthermore, the method may further comprise: additionally obtaining information about an insertion site of the catheter in the patient; and correcting the central venous pressure waveform information according to the catheter insertion site, wherein information about the corrected central venous pressure waveform may be provided to the inference model.

Furthermore, the method may further comprise additionally obtaining at least one of information on the patient's heart rate, information on a pressure state of the lungs or thoracic cavity, information on blood components, and information on a position of the patient on a Frank-Starling curve, and the inference model may perform inference by considering the additionally obtained information together with the central venous pressure waveform information.

According to a third embodiment, a non-transitory computer-readable storage medium stores a computer program, wherein the computer program may be programmed to perform steps comprising: obtaining central venous pressure (CVP) waveform information from a catheter inserted into a patient's body; and obtaining information about stroke volume variation for the patient by providing the central venous pressure waveform information to a pre-trained inference model configured to infer stroke volume variation (SVV) in the heart.

### Effects of the Invention

According to the above embodiments, since information about a CVP waveform is used to infer Stroke Volume Variation (SVV) instead of a conventional static central venous pressure value, more precise inference is possible.

Furthermore, since the aforementioned inference is performed through a deep learning-based model, a result that is robust to noise, etc., which may exist in the corresponding waveform, may be obtained. For example, even though a patient's intra-abdominal pressure may increase due to gas injected into the patient during surgery, and thus the value of the central venous pressure may rise, in an embodiment, the pattern or shape of this waveform, rather than the maximum or minimum value of the central venous pressure waveform, is analyzed by a deep learning model, so that SVV may be accurately inferred even in such a situation where the value is elevated.

Meanwhile, the effects according to an embodiment are not limited to those mentioned above, and other unmentioned technical effects will be clearly understood by a person skilled in the art from the following description.

### BRIEF DESCRIPTION OF THE DRAWINGS

FIG. 1 is a diagram illustrating a configuration of a deep learning-based central venous pressure waveform stroke volume variation inference apparatus according to an embodiment.

FIG. 2 is a schematic diagram illustrating a configuration of a deep learning-based central venous pressure (CVP) waveform stroke volume variation (SVV) inference apparatus according to an embodiment.

FIG. 3 is a flowchart illustrating a process of classifying and analyzing research data according to an embodiment.

FIG. 4 is a diagram illustrating a deep learning-based central venous pressure waveform stroke volume variation inference process according to an embodiment.

### DETAILED DESCRIPTION OF THE INVENTION

Hereinafter, embodiments disclosed in the present specification will be described in detail with reference to the accompanying drawings, but the same or similar components will be assigned the same reference numerals regardless of the drawing numbers, and redundant descriptions thereof will be omitted. The suffixes “module” and “unit” for components used in the following description are given or used interchangeably only for ease of writing the specification, and do not in themselves have distinct meanings or roles. Furthermore, in describing the embodiments disclosed in the present specification, if it is determined that a detailed description of related known art may obscure the gist of the embodiment disclosed in the present specification, the detailed description will be omitted. Furthermore, the accompanying drawings are for the purpose of easy understanding of the embodiments disclosed in the present specification, and the technical spirit disclosed in the present specification is not limited by the accompanying drawings, and it should be understood as including all modifications, equivalents, or substitutes included in the spirit and technical scope of the present disclosure.

Terms including ordinal numbers, such as first, second, etc., may be used to describe various components, but the components are not limited by the terms. The terms are used only to distinguish one component from another.

When a component is referred to as being “connected to” or “coupled to” another component, it should be understood that it may be directly connected or coupled to the other component, but other components may exist in between. On the other hand, when a component is referred to as being “directly connected to” or “directly coupled to” another component, it should be understood that there are no other components in between.

In this application, terms such as “comprise” or “have” are intended to specify the presence of described features, numbers, steps, operations, components, parts, or a combination thereof, and should be understood as not precluding the possibility of the presence or addition of one or more other features, numbers, steps, operations, components, parts, or a combination thereof.

In the present specification, a ‘unit’ includes a unit realized by hardware, a unit realized by software, and a unit realized using both. Furthermore, one unit may be realized using two or more pieces of hardware, and two or more units may be realized by one piece of hardware.

In the present specification, some of the operations or functions described as being performed by a terminal, apparatus, or device may be performed instead by a server connected to the corresponding terminal, apparatus, or device. Similarly, some of the operations or functions described as being performed by a server may also be performed by a terminal, apparatus, or device connected to the corresponding server.

Hereinafter, embodiments will be described in detail with reference to the accompanying drawings.

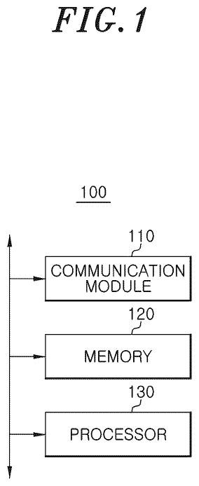

FIG. 1 is a diagram illustrating the configuration of an inference apparatus 100 for inferring stroke volume variation from a central venous pressure waveform according to an embodiment. The configuration of the inference apparatus 100 shown in FIG. 1 is only a simplified example.

A communication module 110 may be configured regardless of its communication method, such as wired and wireless, and may be configured with various communication networks such as a Personal Area Network (PAN) or a Local Area Network (LAN). Furthermore, the communication module 110 may operate based on the well-known World Wide Web (WWW) and may also use a wireless transmission technology used for short-range communication, such as Infrared Data Association (IrDA) or Bluetooth. For example, the communication module 110 may be configured to transmit and receive data necessary for performing the technique according to an embodiment of the present disclosure.

A memory 120 may refer to any storage medium. For example, the memory 120 may include at least one storage medium among a flash memory type, a hard disk type, a multimedia card micro type, a card-type memory (e.g., SD or XD memory, etc.), Random Access Memory (RAM), Static Random Access Memory (SRAM), Read-Only Memory (ROM), Electrically Erasable Programmable Read-Only Memory (EEPROM), Programmable Read-Only Memory (PROM), magnetic memory, a magnetic disk, and an optical disk. The memory 120 may also constitute the database shown in FIG. 1.

The memory 120 may store at least one instruction that may be executed by a processor 130. Furthermore, the memory 120 may store information of any form generated or determined by the processor 130 and information of any form received by a server (200). Furthermore, the memory 120 stores various types of modules, instruction sets, or models.

The processor 130 may perform the technical features according to the embodiments of the present disclosure, to be described later, by executing at least one instruction stored in the memory 120. In one embodiment, the processor 130 may be composed of at least one core and may include a processor for data analysis and/or processing, such as a central processing unit (CPU) of a computer device, a general purpose graphics processing unit (GPGPU), or a tensor processing unit (TPU).

The processor 130 may train a neural network or model designed in a machine learning or deep learning manner. To this end, the processor 130 may perform calculations for training the neural network, such as processing input data for training, extracting features from the input data, calculating errors, and updating the weights of the neural network using backpropagation. Furthermore, the processor 130 may perform inference for a predetermined purpose using a model implemented in an artificial neural network manner.

Hereinafter, an operation or act described as being performed by the inference apparatus 100 is to be understood as being performed by at least one instruction stored in the memory 120 being executed by the processor 130.

First, the inference apparatus 100 may obtain central venous pressure (CVP) waveform information from a catheter inserted into a patient's body. Specifically, a catheter may be inserted into the body of a patient undergoing surgery. At this time, the insertion site of the catheter may vary, and may be, for example, the Superior Vena Cava or a region adjacent to the right atrium.

When a patient's Central Venous Pressure (CVP) waveform is transmitted through such a catheter, a predetermined sensor may be provided to detect such a waveform. Specifically, the sensor may include a pressure transducer and be provided to convert the pressure within the blood vessel into an electrical signal. Such a sensor may be included in the aforementioned inference apparatus 100 or may be provided in a separately provided central venous pressure waveform measurement device.

Meanwhile, the signal detected by the sensor may be converted into a digital signal through an analog-to-digital converter (ADC) according to an embodiment and transmitted to the processor 130.

In an embodiment, the inference apparatus 100, through the processor 130, may collect the received digital signal and obtain central venous pressure waveform data sampled at a constant time interval (e.g., 500 Hz sampling period). The obtained data then may undergo a preprocessing process to perform noise cancellation, baseline correction, or respiratory cycle synchronization, thereby being converted into a form suitable for deep learning-based analysis. Through this, a stable acquisition of CVP, which is the basis for inferring stroke volume variation (SVV), may become possible.

Subsequently, in the inference apparatus 100, the aforementioned preprocessed waveform data may be provided to a pre-trained inference model (or deep learning model) configured to infer stroke volume variation (SVV) in the heart.

Subsequently, in the inference apparatus 100, information about the stroke volume variation for the patient may be obtained. The stroke volume variation information obtained in this way may be used as a key indicator for evaluating the patient's fluid responsiveness.

Here, since this stroke volume variation is inferred or obtained from a deep learning model as described above, a result that is robust to noise, etc., which may exist in the CVP waveform, may be obtained. For example, even though a patient's intra-abdominal pressure may increase due to gas injected into the patient during surgery, and thus the value of the central venous pressure may rise, in an embodiment, the pattern or shape of this waveform, rather than the maximum or minimum value of the central venous pressure waveform, is analyzed by a deep learning model, so that SVV may be accurately inferred even in such a situation where the value is elevated.

Meanwhile, the central venous pressure waveform information may be represented in a numerical type or a graphical format measured over a predetermined time.

If the central venous pressure waveform is provided in a numerical format, the deep learning model at this time may be implemented as an LSTM (Long Short-Term Memory), an RNN (Recurrent Neural Network) series, a BERT (Bidirectional Encoder Representations from Transformers), or a Transformer model, which can receive time-series data as input and process the time-series data by considering its sequence, but is not limited thereto.

Alternatively, if the central venous pressure waveform is provided in an image format, the deep learning model at this time may be implemented as a CNN (Convolutional Neural Network) model specialized for image pattern recognition, but is not limited thereto.

Meanwhile, according to an embodiment, the central venous pressure waveform may be separated into two or more waveforms through a pre-trained waveform decomposition model. Each of these separated two or more waveforms may be provided to the aforementioned deep learning model. In the case of CVP, there is a part affected by respiration and a part affected by heartbeat. Therefore, if the CVP waveform is provided to a pre-trained waveform decomposition model, a cardiac component waveform and a respiratory component waveform for the corresponding CVP waveform may be obtained as a result. Here, since the waveform decomposition model itself may adopt a known model, a description of this part will be omitted. Typically, the performance of a deep learning model varies depending on how the provided input is processed, and the performance may also vary between the case of inputting a single piece of data as it is and the case of separating that single piece of data into two and inputting them. Accordingly, in an embodiment, in inferring SVV, the CVP waveform may be separated into two and provided to the deep learning model, and therefore, more features may be recognized by the corresponding deep learning model, so that inference performance may be improved. To this end, when a waveform decomposition model is adopted, the aforementioned deep learning model may be one that is trained to receive two waveforms, that is, a cardiac component waveform and a respiratory component waveform, as input. For example, when implemented with an LSTM, two time-series data are provided to the input stage, and an SVV may be inferred and output at the output, and supervised learning for this may be performed. That is, the training dataset at this time may be composed of a cardiac component waveform and a respiratory component waveform for the input, and an SVV for the correct answer.

Meanwhile, the aforementioned embodiment may be applied to a patient who is breathing using a predetermined ventilator under positive pressure ventilation. Specifically, the patient (P) may be in a state of breathing while connected to a ventilator that provides positive pressure ventilation. At this time, the inference apparatus 100 collects and analyzes the patient's central venous pressure (CVP) waveform to infer the stroke volume variation (SVV).

At this time, the magnitude and waveform of the change in intrathoracic pressure caused by respiration may vary depending on the type and ventilation mode of the ventilator (e.g., volume-controlled ventilation (VCV), pressure-controlled ventilation (PCV), assist-control (A/C), SIMV, PSV, APRV, etc.), and the setting parameters (tidal volume, respiratory rate, PEEP, inspiratory pause, rise time, etc.). Accordingly, the patient's SVV may also vary depending on the type, mode, and setting values of the ventilator.

Accordingly, in an embodiment, information about the ventilator, for example, the manufacturer, as well as the product name, operating mode, or setting values, may be provided to the inference model. That is, even for the same CVP waveform, the SVV may be inferred differently depending on the type of ventilator. To this end, the aforementioned deep learning model may be trained to receive this metadata as input, and may be driven by receiving this metadata as input during the actual inference process.

In an embodiment, the inference apparatus 100 may additionally obtain information about the insertion site of the catheter in the patient through the processor 130. The insertion site of the catheter may affect the stability and clinical interpretability of the CVP waveform. Specifically, a position near the right atrium in the Superior Vena Cava (SVC) is considered an ideal insertion location. In this case, the obtained CVP waveform is stable and has low noise, and well reflects a clinically meaningful cardiovascular state. On the other hand, if the catheter tip has entered into the right atrium, it directly reflects the intra-atrial pressure, and the waveform may be exaggerated or may include abnormal noise. In such a case, the accuracy of stroke volume variation (SVV) inference may be degraded due to waveform distortion.

Furthermore, if the catheter is located too superiorly (towards the periphery) within the superior vena cava, it may be measured at a value lower than the actual right atrial pressure, making it difficult to obtain a clinically meaningful pressure waveform.

Therefore, in an embodiment, the inference apparatus 100 may correct the CVP waveform by additionally considering the catheter insertion site information. Furthermore, the corrected CVP waveform may be provided to the aforementioned deep learning model.

At this time, the correction method may be various. For example, the amplitude, phase, baseline, etc., of the CVP waveform may be varied by correction according to the insertion site. Here, how much to correct may be defined by rule-based software. For example, a rule may be given in advance such that if the insertion site of the catheter is within a certain distance from the right atrium in the superior vena cava, it is not corrected, but if it is superior to a predetermined point within the superior vena cava, the amplitude is reduced by 10%, the phase is increased by 10 degrees, and the baseline is reduced by 10%.

In an embodiment, the inference apparatus 100 may additionally obtain at least one of information on the patient's heart rate or respiratory rate, information on a pressure state of the lungs or thoracic cavity, information on blood components, and information on a position of the patient on a Frank-Starling curve. Specifically, the inference apparatus 100, in addition to the central venous pressure (CVP) waveform, may additionally obtain at least one of information on heart rate, information on the pressure state of the lungs or thoracic cavity, information on blood components, and information on the position of the patient on the Frank-Starling curve, in order to more precisely evaluate the patient's hemodynamic state.

The information obtained in this way may be provided to the aforementioned deep learning model together with the CVP waveform. That is, even for the same CVP waveform, the SVV may be inferred differently depending on the aforementioned information, for example, blood components, heart rate, lung capacity, etc. To this end, the aforementioned deep learning model may be trained to receive this information as input, and may be driven by receiving this metadata as input during the actual inference process.

Here, the relative position on the Frank-Starling curve, which represents the relationship between the patient's cardiac filling pressure and stroke volume, is known to be crucial for determining fluid responsiveness. Accordingly, if the patient's position on the ascending limb, plateau, or saturation part of the curve is estimated by synthesizing clinical data such as central venous pressure, cardiac output (CO), and ejection fraction (EF), that information may be provided as an auxiliary input to the aforementioned deep learning model.

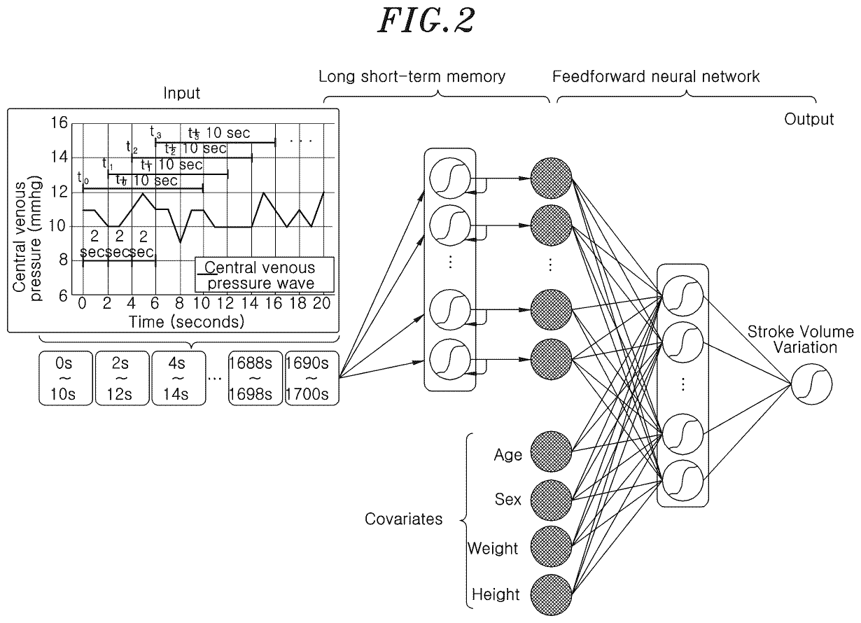

FIG. 2 is a diagram conceptually illustrating an operating method or architecture of a deep learning-based central venous pressure (CVP) waveform stroke volume variation (SVV) inference apparatus according to an embodiment. As shown in FIG. 2, the inference apparatus 100, in order to infer a patient's stroke volume variation (SVV) by receiving a central venous pressure waveform as input, may include a structure combining a Long Short-Term Memory (LSTM) or a Feedforward Neural Network (FFN), but is not limited thereto.

First, FIG. 2 shows a central venous pressure (CVP) waveform for a patient as ‘Input’. This waveform may be sampled from the CVP waveform over a predetermined time interval (e.g., 10 seconds) at a regular interval (e.g., 2 seconds), and as a result, may be configured as time-series input data. The figure illustrates continuous window data along the time axis, such as 0 to 10 seconds, 2 to 12 seconds, 4 to 14 seconds, etc. This input CVP waveform is provided to an LSTM module, and the temporal correlations and periodic variation patterns within the waveform are learned. The LSTM module captures changes due to heartbeat and respiration from the continuous CVP waveform sections and outputs a high-dimensional feature vector that may reflect the stroke volume variation.

Furthermore, referring to FIG. 2, covariates may be provided as additional input to reflect the physiological differences of the patient according to an embodiment. The covariates include clinical data such as the patient's age, sex, weight, and height, which are used as auxiliary inputs along with the waveform-based signal to increase inference accuracy. The output of the LSTM module and the covariate vector are input to a Feedforward Neural Network (FFN). The FFN extracts comprehensive features through connections between each input node and finally performs regression inference on the patient's stroke volume variation (SVV) as a continuous value.

Therefore, as shown in FIG. 2, the inference apparatus 100 implements a deep learning-based SVV inference apparatus that enables non-invasive and accurate fluid responsiveness evaluation by comprising (i) an LSTM structure that reflects the time-series information of the central venous pressure waveform, (ii) a covariate input that reflects the individual characteristics of the patient, and (iii) a feedforward neural network that finally integrates the two types of input to calculate the SVV.

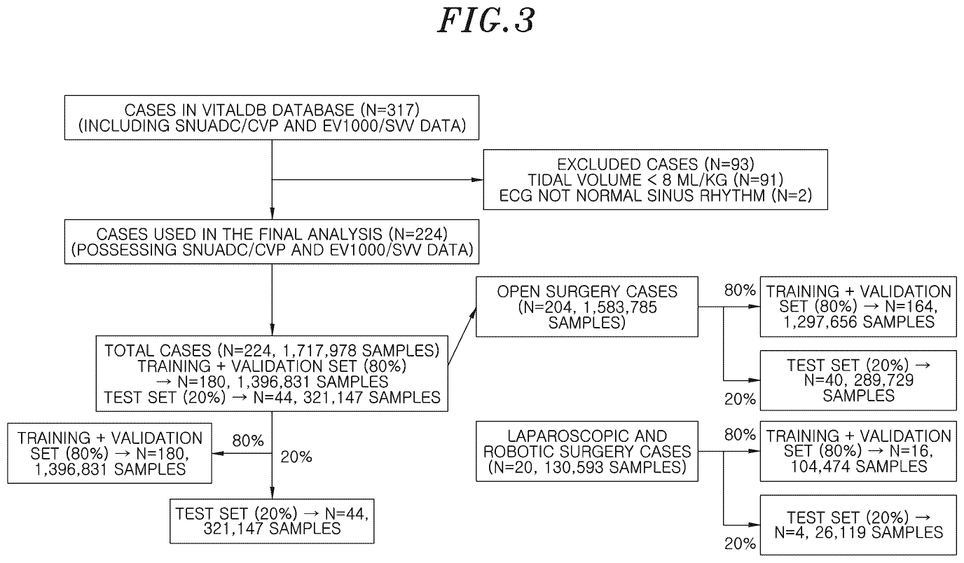

FIG. 3 is a flowchart illustrating a process of classifying and analyzing research data according to an embodiment. As shown in FIG. 3, the analysis herein is based on a total of 317 cases including SNUADC/CVP and EV1000/SVV data collected from the Vital DB database. Among these, 224 cases were used for the final analysis, excluding cases with a tidal volume of less than 8 mL/kg (n=91) and electrocardiogram (ECG) cases that were not normal sinus rhythm (n=2). The 224 cases consist of a total of 1,717,978 samples, of which 80% were divided into a training and validation set (n=180, 1,396,831 samples) and 20% into a test set (n=44, 321,147 samples). Furthermore, the cases were subdivided according to the type of surgery. For open surgery cases (n=204, 1,583,785 samples), they were divided into a training and validation set (n=164, 1,297,656 samples) and a test set (n=40, 289,729 samples). For laparoscopic and robotic surgery cases (n=20, 130,593 samples), they were divided into a training and validation set (n=16, 104,474 samples) and a test set (n=4, 26,119 samples). This analysis demonstrates that the reliability and generalization of deep learning-based stroke volume variation (SVV) inference may be secured through the process of training, validating, and testing central venous pressure waveform data collected in various surgical environments.

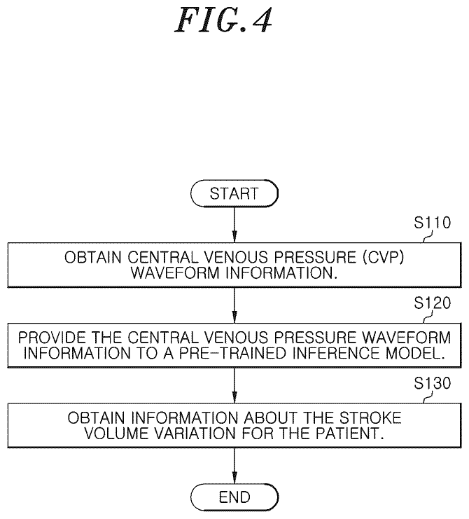

Hereinafter, FIG. 4 will be described. The method shown in FIG. 4 may be performed by a deep learning-based central venous pressure waveform stroke volume variation inference apparatus 100 including a processor 130.

Meanwhile, FIG. 4 is merely exemplary, and the spirit of the present disclosure is not to be construed as being limited to that shown in FIG. 4. For example, the steps may be configured in a different order from that shown in FIG. 4, at least one of the steps shown in FIG. 4 may not be performed, or one or more steps not shown in FIG. 4 may be additionally performed.

Hereinafter, the deep learning-based central venous pressure waveform stroke volume variation inference method will be described in order. Since the operation (function) of the deep learning-based central venous pressure waveform stroke volume variation inference method according to an embodiment is essentially the same as the function of the deep learning-based central venous pressure waveform stroke volume variation inference apparatus, redundant descriptions with FIGS. 1 to 3 will be omitted.

FIG. 4 is a diagram illustrating a deep learning-based central venous pressure waveform stroke volume variation inference process according to an embodiment. Referring to FIG. 4, in step S110, central venous pressure (CVP) waveform information is obtained from a catheter inserted into a patient's body. In step S120, the central venous pressure waveform information is provided to a pre-trained inference model configured to infer stroke volume variation (SVV) in the heart. In step S130, information about the stroke volume variation for the patient is obtained.

Meanwhile, the methods according to the various embodiments described above may be implemented in the form of an application or software program that may be installed on an existing electronic device.

Furthermore, all or part of the method may be configured as a plurality of software function modules and implemented in an operating system (OS). Alternatively, each step may be configured as one software function module, or the steps may be combined to form one software function module and implemented on an operating system. Therefore, even if not all of some embodiments of the present disclosure are implemented as a single software function module, if a plurality of software function modules implement each step of the present disclosure, and the plurality of software function modules are implemented in one operating system, it can be understood that the method of the present disclosure has been implemented.

Furthermore, the methods according to the various embodiments described above can be implemented even by only a software upgrade or a hardware upgrade to an existing electronic device. Furthermore, the various embodiments described above can also be performed through an embedded server provided in an electronic device or an external server of the electronic device.

Meanwhile, according to an embodiment, the various embodiments described above may be implemented as software including instructions stored on a computer-readable storage medium using software, hardware, or a combination thereof. In some cases, the embodiments described in this specification may be implemented by the processor itself. According to a software implementation, embodiments such as the procedures and functions described in this specification may be implemented as separate software modules. Each of the software modules can perform one or more of the functions and operations described in this specification.

Meanwhile, a computer or a similar device is a device that can call instructions stored from a storage medium and operate according to the called instructions, and may include the device according to the disclosed embodiments. When the command is executed by a processor, the processor may perform the function corresponding to the command directly, or by using other components under the control of the processor. The command may include code generated or executed by a compiler or interpreter.

A device-readable storage medium may be provided in the form of a non-transitory computer-readable storage medium. Here, ‘non-transitory’ only means that the storage medium does not include a signal and is tangible, and does not distinguish between whether data is stored semi-permanently or temporarily in the storage medium. At this time, a non-transitory computer-readable medium refers to a medium that stores data semi-permanently and is readable by a device, not a medium that stores data for a short period of time, such as a register, cache, or memory. Specific examples of a non-transitory computer-readable medium may include a CD, DVD, hard disk, Blu-ray disc, USB, memory card, ROM, and the like.

As described above, exemplary embodiments have been disclosed in the drawings and the specification. Although the embodiments have been described using specific terms in this specification, they are used only for the purpose of explaining the technical spirit of the present disclosure and are not used to limit the meaning or the scope of the present disclosure described in the claims. Therefore, a person having ordinary skill in the art will understand that various modifications and other equivalent embodiments are possible therefrom. Accordingly, the true technical scope of the present disclosure should be determined by the technical spirit of the appended claims.

What is claimed is:

1. A stroke volume variation inference apparatus, comprising:
a memory storing at least one instruction; and
a processor,
wherein as the at least one instruction is executed by the processor, the processor is configured to:
obtain central venous pressure (CVP) waveform information from a catheter inserted into a patient's body, and
obtain by providing the central venous pressure waveform information to a pre-trained inference model configured to infer stroke volume variation (SVV) in a heart, such that information about the stroke volume variation for the patient.

2. The stroke volume variation inference apparatus of claim 1, wherein the central venous pressure waveform information is a central venous pressure measured over a predetermined time, represented in a numerical type or a graphical format.

3. The stroke volume variation inference apparatus of claim 1,
wherein as the at least one instruction is executed by the processor,
information about the central venous pressure waveform is provided to a pre-trained waveform decomposition model,
a cardiac component waveform and a respiratory component waveform, which are results of decomposing the central venous pressure waveform information, are obtained from the waveform decomposition model, and
the cardiac component waveform and the respiratory component waveform are provided to the inference model as the central venous pressure waveform information.

4. The stroke volume variation inference apparatus of claim 1,
wherein the patient is a patient breathing using a predetermined ventilator under positive pressure ventilation, and
the inference model infers the information about the stroke volume variation for the patient by further considering information about a type of ventilator the patient is using for respiration.

5. The stroke volume variation inference apparatus of claim 1,
wherein as the at least one instruction is executed by the processor,
information about an insertion site of the catheter in the patient is additionally obtained,
the central venous pressure waveform information is corrected according to the catheter insertion site, and
information about the corrected central venous pressure waveform is provided to the inference model.

6. The stroke volume variation inference apparatus of claim 1,
wherein as the at least one instruction is executed by the processor,
at least one of information on the patient's heart rate or respiratory rate, information on a pressure state of lungs or a thoracic cavity, information on blood components, and information on a position of the patient on a Frank-Starling curve is additionally obtained, and
the inference model performs inference by considering the additionally obtained information together with the central venous pressure waveform information.

7. A stroke volume variation inference method to be performed by a stroke volume variation inference apparatus, the method comprising:
obtaining central venous pressure (CVP) waveform information from a catheter inserted into a patient's body; and
obtaining information about stroke volume variation for the patient by providing the central venous pressure waveform information to a pre-trained inference model configured to infer stroke volume variation (SVV) in the heart.

8. The stroke volume variation inference method of claim 7, wherein the central venous pressure waveform information is a central venous pressure measured over a predetermined time, represented in a numerical type or a graphical format.

9. The stroke volume variation inference method of claim 7,
the method further comprising:
providing the central venous pressure waveform information to a pre-trained waveform decomposition model; and
obtaining a cardiac component waveform and a respiratory component waveform, which are results of decomposing the central venous pressure waveform information, from the waveform decomposition model,
wherein the cardiac component waveform and the respiratory component waveform are provided to the inference model as the central venous pressure waveform information.

10. The stroke volume variation inference method of claim 7,
wherein the patient is a patient breathing using a predetermined ventilator under positive pressure ventilation, and
the inference model infers the information about the stroke volume variation for the patient by further considering information about a type of ventilator the patient is using for respiration.

11. The stroke volume variation inference method of claim 7,
the method further comprising:
additionally obtaining information about an insertion site of the catheter in the patient; and
correcting the central venous pressure waveform information according to the catheter insertion site,
wherein information about the corrected central venous pressure waveform is provided to the inference model.

12. The stroke volume variation inference method of claim 7,
the method further comprising:
additionally obtaining at least one of information on the patient's heart rate, information on a pressure state of lungs or a thoracic cavity, information on blood components, and information on a position of the patient on a Frank-Starling curve,
wherein the inference model performs inference by considering the additionally obtained information together with the central venous pressure waveform information.

13. A non-transitory computer-readable storage medium storing a computer program,
wherein the computer program is programmed to perform a method comprising:
obtaining central venous pressure (CVP) waveform information from a catheter inserted into a patient's body; and
obtaining information about stroke volume variation for the patient by providing the central venous pressure waveform information to a pre-trained inference model configured to infer stroke volume variation (SVV) in the heart.
